(19) United States
(12) Patent Application Publication
(10) Pub. No.: US 2024/0252509 A1
(43) Pub. Date: Aug. 1, 2024

(54) SELF-PRESERVING COMPOSITIONS AND MULTI-USE DISPENSERS FOR ADMINISTERING ALPHA-1062

(71) Applicant: ALPHA COGNITION INC., Vancouver, British Columbia (CA)

(72) Inventor: Fred D. SANCILIO, Stuart, FL (US)

(21) Appl. No.: 18/560,636

(22) PCT Filed: May 14, 2021
(86) PCT No.: PCT/CA2021/050666

§ 371 Date: Nov. 13, 2023

(51) Int. Cl. A61K 31/55; A61K 9/00; A61P 31/04

(52) U.S. Cl. CPC A61K 31/55; A61K 9/006; A61P 31/04

(57) ABSTRACT

A self-preserving anti-microbial pharmaceutical composition, including a compound Alpha-1062 or salt thereof, wherein the composition is essentially absent of additional antimicrobial preservatives. In some examples, the compositions are in the form of a liquid. Also disclosed is a multi-use dispenser configured for transmucosal administration of a pharmaceutical composition in the form of a liquid including Alpha-1062 or salt thereof and Alpha-1062 or salt thereof for use in the treatment of neurological disease in a subject, wherein said subject suffers additionally from a microbial infection, or for use in treating a microbial infection. Also disclosed is a method of killing a microorganism or a method for disinfecting, e.g., disinfecting a pharmaceutical composition, including contacting a microorganism with Alpha-1062 or salt thereof.

The present invention is in the field of compositions, formulations and dispensing devices for pharmaceutical agents, in addition to medical and/or anti-microbial methods.

The invention relates to a self-preserving anti-microbial pharmaceutical composition, comprising a compound Alpha-1062 or salt thereof, wherein the composition is essentially absent of additional antimicrobial preservatives. The invention relates preferably to such compositions in the form of a liquid. In another aspect the invention relates to a multi-use dispenser configured for transmucosal administration of a pharmaceutical composition in the form of a liquid comprising Alpha-1062 or salt thereof.

In another aspect, the invention relates to Alpha-1062 or salt thereof for use in the treatment of neurological disease in a subject, wherein said subject suffers additionally from a microbial infection, or for use in treating a microbial infection. In another aspect, the invention relates to a method of killing a microorganism or a method for disinfecting, e.g. disinfecting a pharmaceutical composition, comprising contacting a microorganism with Alpha-1062 or salt thereof.

### BACKGROUND OF THE INVENTION

Alzheimer's disease (AD) is the most common form of dementia in the elderly. It is characterized by progressive memory loss, with impairment of attentiveness, semantic memory, abstract thinking and other cognitive functions. Several experimental therapies having potential of disease modification are currently undergoing investigation, with the most prominent involving antibodies targeting abnormal accumulation of proteins such as extracellular beta amyloid oligomers and plaques, and intracellular tau protein. However, recent phase III clinical studies of antibody therapies targeting beta-amyloid have failed to show sufficient therapeutic efficacy.

Another early marker of AD is the increasing loss of cholinergic neurons and reduced density of nicotinic acetylcholine receptors (nAChRs) in the course of the disease. Cholinergic enhancement is therefore considered a symptomatic therapy to improve cognitive function through enhancement of cholinergic transmission. Drugs licensed for this purpose are tacrine, donepezil, rivastigmine and galantamine, namely inhibitors of the enzyme acetylcholinesterase (AChE) and to a varying extent, butyryl cholinesterase (BuChE), which normally metabolize and thereby inactivate the cholinergic transmitter, acetylcholine (ACh). The enhancement of cholinergic function in the brain resulting from the action of these drugs enhances cognition and improves various behavioral aspects in AD.

Galantamine is a tertiary amide, belonging to the phenanthrene chemical class, which occurs naturally in some bulb plants. In addition to inhibition of AChE, galantamine also enhances cholinergic activity by non-competitive, allosteric modulation of the nAChR. It was introduced as a drug for AD in 2000 and now is approved in more than 70 countries. The indication is generally ‘mild to moderate dementia of the Alzheimer's type’. It is currently marketed as Razadyne® in the USA, and as Reminyl® elsewhere.

Similar to other cholinesterase inhibitors, galantamine has a clinically significant level of gastro-intestinal (GI) side effects, including nausea, vomiting and diarrhea. To accommodate patients, cholinesterase inhibitors are often initially administered at a low (non-efficacious) dose, and then adjusted to what the patients experience as an acceptable level of GI side effects, making it likely that most, if not all, patients never achieve treatment with the most therapeutically effective levels.

To enhance the lipophilicity of acetylcholinesterase inhibitors and improve their passage through mucosal tissue, hydrophobic side chains have been appended to the basic alkaloid structures.

Galantamine derivatives and pro-drugs are described in EP 1940817, WO 2009/127218 and US 2009/0253654.

The galantamine pro-drug Alpha-1062, is a benzoic ester of galantamine ((4aS,6R,8aS)-5,6,9,10,11,12-hexahydro-3-methoxy-11-methyl-4aH-[1]benzofuro[3a,3,2-ef][2]benzazepin-6-ol benzoate). It was developed to enhance the hydrophobicity of galantamine. Alpha-1062 exhibits essentially no pharmacological activity until it is cleaved by a carboxyesterase, resulting in the release of galantamine.

WO 2014/016430 discloses transmucosal administration of Alpha-1062 via intranasal, buccal or sublingual modes, in addition to various formulations and salts of Alpha-1062, including for example lactate, gluconate, maleate and saccharate salts. The Alpha-1062 gluconate salts described in WO 2014/016430 show solubility in water above 10% weight per volume (w/v) and are therefore suitable for intranasal or other liquid, preferably solution, pharmaceutical formulations.

The first nasal spray pumps were developed approximately 50 years ago and replaced the previous step by step droppers and pipettes. Nasal spray pumps are now widely used to moisturize the nasal mucosa using saline solutions, as nasal preparations for the administration of topically acting drugs, e.g. nasal decongestants, or for the non-invasive administration of substances which need to reach systemic circulation, e.g. anti-migraine medication or hormones (Marx and Birkhoff, “Multi-Dose Container for Nasal and Ophthalmic Drugs: A Preservative Free Future?” in Drug Development—A Case Study Based Insight into Modern Strategies, ed. Chris Rundfeldt, 2011).

For many disease indications, multi-dose devices are cost effective and convenient means to provide the safety and precision in administration of active agents that regulatory bodies require. However, until now, most medications administered transmucosally, commonly as solutions, emulsions or suspensions, contain a preservative to support long storage times and proper in-use stability for multi-use or multi-dose dispensers.

Despite the advantages of nasal administration, the use of preservatives in nasal sprays is controversial. Reports suggest that preservatives in nasal sprays might increase the risk of adverse events for patients. For instance, reports have suggested that benzalkonium chloride (BAC), commonly used in nasal decongestants, can cause ciliotoxicity (impaired ciliary activity) of the nasal mucosa and therefore nasal irritation (Graf, “Adverse effects of benzalkonium chloride on the nasal mucosa: allergic rhinitis and rhinitis medicamentosa”, Clin Ther. 1999 October; 21(10): 1749-55, and Riechelmann et al, “Nasal toxicity of benzalkonium chloride”, Am J Rhinol. 2004; 18(5):291-9). In other trials employing nasal sprays with BAC, various adverse effects have been observed. These side effects can include increased mucosal swelling and nasal hyper-reactivity, type IV hypersensitivity, decrease of mucociliary clearance, and nasal mucosa dysplasia.

As a further complication, patient medication adherence represents a significant challenge in subjects with neurological disease. Impairment of cognitive functions and dementia, in particular, may substantially compromise drug compliance and adherence behavior. Medication adherence is likely to be improved by reducing the frequency and number of medications and improving simplicity in delivery modes for elderly or demented patients (Arlt et al., Adherence to Medication in Patients with Dementia, Drugs Aging 2008; 25 (12): 1033-1047).

Although patch formulations have been proposed for delivery of acetylcholinesterase inhibitors in order to improve patient compliance (Winblad et al, Caregiver preference for rivastigmine patch relative to capsules for treatment of probable Alzheimer's disease. Int J Geriatr Psychiatry 2007; 22: 485-91), the delivery of Alpha-1062 via the transmucosal route, preferably via nasal, sub-lingual or buccal routes, and associated enhanced bioavailability appears preferred over topical administration options.

Preservatives may therefore cause nasal irritation and compliance can be improved when irritation is reduced. Patches are also commonly removed by patients, which represents a further hurdle to treatment compliance in patients with cognitive disorders.

Despite recent developments with respect to formulating Alpha-1062, further improvements are required in providing efficient, simple-to-use means for administering medications for treating neurological disease that show low side effects and good patient compliance, especially in subjects with cognitive difficulties.

### SUMMARY OF THE INVENTION

In light of the prior art the technical problem underlying the invention was the provision of improved or alternative means for formulating and/or preparing medication for treating neurological disease, preferably Alpha-1062 or salts thereof, in a simple and easy-to-use form that shows low side effects and good patient compliance.

Another objective of the invention was to develop improved or alternative formulations or doses for neurological disease medication, that are simple and easy-to-use, with low side effects and good patient compliance, with improved storage properties.

This problem is solved by the features of the independent claims. Preferred embodiments of the present invention are provided by the dependent claims.

The invention therefore relates to a self-preserving anti-microbial pharmaceutical composition, comprising a compound Alpha-1062 or salt thereof, wherein the composition is essentially absent of additional antimicrobial preservatives.

In one embodiment, the composition is in the form of a liquid.

The invention therefore also relates to a self-preserving anti-microbial pharmaceutical composition in the form of a liquid, comprising Alpha-1062 or salt thereof, wherein the composition is essentially absent of additional antimicrobial preservatives.

The invention therefore also relates to a self-preserving anti-microbial pharmaceutical composition in the form of a solution, comprising Alpha-1062 or salt thereof, wherein the composition is essentially absent of additional antimicrobial preservatives.

The invention therefore also relates to a self-preserving anti-microbial pharmaceutical composition in the form of an emulsion or suspension, comprising Alpha-1062 or salt thereof, wherein the composition is essentially absent of additional antimicrobial preservatives.

Various liquid compositions, in addition to solutions, are contemplated, such as emulsions, suspensions, and the like. In one embodiment, the liquid composition comprises a sufficient amount or volume of composition for multiple administration events (doses) to a subject.

As described in detail below, the invention is based on the surprising identification of anti-microbial properties of Alpha-1062. No indication is evident in the prior art prior that this agent has anti-microbial properties. As is described at length in the introduction above, Alpha-1062 was known to exhibit an effect against cognitive impairment by acting as a pro-drug of galantamine. No suggestion is evident in the art that galantamine or its prodrug Alpha-1062 could exhibit the property of actively reducing CFU/mL of pathogenic bacteria, yeast and fungi, as demonstrated according to the USP 51 test.

The invention therefore also relates to the use of Alpha-1062 or salt thereof as a preservative in a pharmaceutical composition. The invention therefore also relates to a composition comprising Alpha-1062 or salt thereof, wherein the composition is in the form of a solution and a sufficient quantity is present to enable multiple doses to a subject. The invention therefore also relates to compositions comprising Alpha-1062 or salt thereof in a solution configured for multi-dose administration to a subject in need thereof.

The surprising finding of the antimicrobial properties of Alpha-1062 enables various options for formulating Alpha-1062 and various novel applications of the molecule that were not previously thought possible, including, without limitation, multi-dose and/or multi-use dispensers of Alpha-1062 solutions that are essentially free of additional preservatives.

The identification of this novel property (anti-microbial effect) of a known substance (Alpha-1062) opens new clinical perspectives and creates a new clinical situation when considering either formulating or administering the substance. New patient populations have been enabled due to the discovery of the novel properties, and new dosage regimes and formulation options may now be employed, based on the surprising property.

The provision of a self-preserving Alpha-1062 composition, preferably as a liquid, or more preferably as an emulsion or solution, such as in the form of multi-use dispensers without additional preservatives, enables reduced side effects, for example side effects caused by nasal administration of a solution that comprises an additional preservative. Storage properties of any given formulation may now be improved, i.e. longer storage times may be evident due to the discovery of the anti-microbial properties.

In some embodiments, reduced side effects may be achieved. Such reduced side effects may relate to nasal irritation, such as, but without limitation to, increased mucosal swelling and nasal hyper-reactivity, type IV hypersensitivity, decrease of mucociliary clearance, and nasal mucosa dysplasia.

In one embodiment, the Alpha-1062 solution comprises no further preservative. In one embodiment, the composition of the invention does not comprise benzalkonium chloride (BAC).

In some embodiments, the composition of the invention does not comprise one or more, preferably does not comprise any, additional preservative(s), selected from the list consisting of benzalkonium (preferably benzalkonium chloride), benzyl alcohol, thimerosal (merthiolate), edetate disodium, monobasic sodium phosphate, providone, dibasic sodium phosphate, disodium eta, potassium phosphate monobasic, iodine, phenylcarbinol and sodium silicoaluminate.

Furthermore, the provision of a self-preserving Alpha-1062 solution, such as in the form of multi-use dispensers without additional preservatives, enables good patient compliance compared to single-use or single-dose intranasal administration, as described in the art. The provision of a simple nasal spray dispenser, capable of multiple uses, but without preservatives, is a simple and low side effect-option for patients. Firstly, low nasal irritation should lead to enhanced patient compliance, as discomfort in the nose is reduced. Secondly, keeping a single dispenser over time, for multiple uses, makes administration simpler for elderly subjects. In contrast, the daily use of single-dose dispensers is associated with additional cost and complication, requiring a (potentially senile or demented) patient to continually open, use and dispose of single dose dispensers, leading to complications and delivery burden.

In one embodiment, the self-preserving composition comprises a sufficient amount of Alpha-1062 to have an anti-microbial effect.

In one embodiment, the self-preserving pharmaceutical composition is characterized in that the compound Alpha-1062 or salt thereof is present at a concentration of 1 to 200 mg/mL, preferably between 5 and 100 mg/mL.

In some embodiments, the compound Alpha-1062 is present at a concentration of 1 to 500 mg/mL, preferably between 1 and 400 mg/mL, more preferably between 1 and 300 mg/mL, preferably between 1 and 200 mg/mL, preferably between 5 and 100 mg/mL.

In some embodiments, the Alpha-1062 is present at about 5 mg/mL, or 10, 15, 20, 25, 30, 35, 40, 45, 50, 55, 60, 65, 70, 75, 80, 85, 90, 95, 100, 110, 120, 130, 140, or at about 150 mg/mL. Ranges constructed form any given of the afore-mentioned values are also contemplated.

In some embodiments, the Alpha-1062 is present as a salt, preferably a lactate, gluconate, maleate or saccharate salt. Generation of Alpha-1062 salts are described in the art, e.g. in WO 2014/016430, and can be carried out without undue burden.

In some embodiments, the salt comprises stoichiometric and/or non-stoichiometric salts and/or hydrates of the chemical substances according to Alpha-1062, whereby the salt is preferably described as:

Alpha-1062·nHX·mH2O,
wherein n and m=0-5, and n and m can be the same or different, and HX is an acid, selected preferably from lactic acid, gluconic acid, maleic acid or saccharic acid.

In some embodiments, other acids may be employed for the Alpha-1062 salt formation.

Acids useful for preparing the pharmaceutically acceptable acid addition salts according to the invention include inorganic acids and organic acids, such as sulfamic, amidosulfonic, 1,2-ethanedisulfonic, 2-ethylsuccinic, 2-hydroxyethanesulfonic, 3-hydroxynaphthoic, acetic, benzoic, benzenesulfonic acid, carboxylic, ethylenediamine tetraacetic acid, camphorsulfonic, citric, dodecylsulfonic, ethanesulfonic, ethenesulfonic, ethylenediamine tetraacetic, fumaric, glubionic, glucoheptonic, gluconic, glutamic, hexylresorcinic, hydrobromic, hydrochloric, isethionoc, (bi)carbonic, tartaric, hydriodic, lactic, lactobionic, laevulinic, laurylsulfuric, lipoic, malic, maleic, malonic, mandelic, methanesulfonic, mucic, naphthalenesulfonic, nitric, oxalic, pamoic, pantothenic, perchloric, phosphoric, polygalacturonic, pectic, propionic, salicylic, succinic or sulfuric acid, p-tuluenesulfonic, wherein hydrochloric, hydrobromic, sulfuric, nitric, phosphoric and perchloric acids, as well as tartaric, citric, acetic, succinic, maleic, fumaric and oxalic acids.

In one embodiment, the Alpha-1062 salt has solubility in water of at least 10%, preferably >20%, or more preferably >30% weight per volume (w/v). The high solubility enables higher concentrations of the compound to be administered in smaller volumes, thereby further enhancing administration via e.g. transmucosal administration.

The preferred transmucosal administration represents a beneficial mode of delivery due to a combination of factors. The enhanced solubility of Alpha-1062 salts allows higher concentrations of Alpha-1062 to be administered, thereby enabling larger amounts of the active substance after cleavage (galantamine) to be active in the brain. The prodrug properties of Alpha-1062 are exploited and enhanced by the transmucosal application of the Alpha-1062 salts.

In one embodiment, the self-preserving pharmaceutical composition of the invention has the compound Alpha-1062 present as a gluconate salt, preferably at a concentration of 50-100 mg/mL, more preferably 70-90 mg/mL, or alternatively at a concentration disclosed herein. A skilled person is aware of varying the concentration of the active agent according to common practice.

In one embodiment, the self-preserving pharmaceutical composition of the invention is characterized in that the compound Alpha-1062 is present at a concentration sufficient to reduce 1×10⁴ to 1×10⁶ colony-forming units per mL (CFU/mL) of Escherichia coli, Pseudomonas aeruginosa, Staphylococcus aureus, Candida albicans and/or Aspergillus brasiliensis to <100 CFU/mL, preferably <10 CFU/mL, within 14 days treatment, according to the United States Pharmacopeia Chapter 51 preservative test (USP 51).

As can be derived from the examples below, the anti-microbial activity against various pathogenic microbes has been shown using an 82 mg/mL Alpha-1062 gluconate solution. However, a skilled person is capable of adjusting both the salt anion (depending on the acid used in salt formation) and concentration of Alpha-1062 or salt thereof to find a composition with essentially the same, enhanced, or reduced but still practically useful, anti-microbial properties, based on the USP 51 test.

In some embodiments, the liquid composition is stable, i.e. exhibits sufficient drug or pro-drug stability and low microbial loads, over extended periods. In some embodiments, the extended period is for about 1 week, or 1, 2, 3, 4, 5, 6, 7, 8 weeks, or longer. In some embodiments, the composition is stable for about 1 month, or 2, 3, 4, 5, 6, 7, 8, 9, 10, 11, or 12 months. During this time, the composition may be administered via multi-use device of the invention, or in storage. In some cases, storage of the composition is possible (preferably without use by a subject) for about 1, 2, 3, 4 or 5 years.

In some embodiments, the liquid compositions are configured for any mode of pharmaceutical administration in which a liquid can be effectively delivered to a subject. For example, transmucosal administration forms, such as nasal, buccal or sub-lingual, or other mode employing administration to the oral or nasal cavity are envisaged. For example, injections, such as parenteral, intravenous, intrathecal, intradermal, subcutaneous and/or intramuscular are considered.

In some embodiments, the liquid formulations are configured for any one or more of the above administration modes. A skilled is aware of technical means employed in configuring compositions for specific modes of administration. For example, a composition configured for injection may be formulated, packaged, or prepared in a different manner from compositions prepared for oral delivery.

A further aspect of the invention relates to a multi-use dispenser configured for transmucosal administration of a pharmaceutical composition in the form of a liquid, comprising a self-preserving anti-microbial solution as described herein with Alpha-1062 or salt thereof.

In this aspect, in the non-limiting context of a multi-use dispenser, the invention therefore combines the properties, features and advantages of multi-use transmucosal delivery devices with the anti-microbial property of Alpha-1062 or salts thereof.

Multi-use dispensers are known to those skilled in the art and may come in the form of any device suitable for multiple doses or multiple applications of the compound. In other words, it is preferred that a multi-use device does not require opening or re-filling between multiple administration events and is typically stable, with sufficient API stability and low microbial loads, over extended periods. In some embodiments, the dispenser may be suitable for administration of any given liquid formulation, preferably a solution, although emulsions and suspensions, and the like, are contemplated.

Liquid formulations are known to the skilled person and are common in pediatric or geriatric patient populations. A liquid formulation typically requires a stable, dissolved, or suspended form of the compound that meets release, bioavailability, and taste/irritation requirements. Both immediate and sustained release liquid products are contemplated. Liquid formulation strategies typically include direct incorporation of an active drug or pro-drug in a dissolved or suspended or emulsified state. Alternative states include incorporation of an active drug or pro-drug in the form of a suspended drug-ion exchange resin complex, or incorporation of drug or pro-drug in the form of a dissolved or suspended drug-cyclodextrin inclusion complex, or in an emulsified state.

Dispensers configured for transmucosal administration are also known in the art, as are transmucosal administration routes, which relate preferably to oral, nasal, vaginal, and urethral modes. The transmucosal membranes are relatively permeable, have a rich blood flow and hence allow the rapid uptake of a drug into systemic circulation to avoid first pass metabolism. The oral transmucosal delivery preferably relates to the buccal and sublingual routes. This route of drug delivery offers a number of benefits over other drug delivery approaches and allows drugs to circumvent some of the body's natural “defense mechanisms” like first pass metabolism, the harsh stomach environment, and potentially metabolism in the intestines due to exposure to microbial populations present in the intestines. By way of example, several approaches have been used like drug delivery through the nasal route by using sprays, pumps and gels while the mucoadhesive, quick dissolve tablets and solid lozenge formulations are suitable for the oral mucosal route, also, vaginal or urethral routes can be explored using mucoadhesive suppositories, in-situ gel and foam formulations.

In one embodiment, a therapeutically effective amount of the compound is administered using a suitable metered multi-use dose device or dispenser, such as a multi-use atomizer, multi-use sprayer, multi-use pump spray, multi-use dropper, multi-use squeeze tube or bottle, multi-use metered dose device or multi-use nasal sprayer or inhaler.

In one embodiment, a therapeutically effective amount of the compound is administered under the tongue (sub-lingual) by dispensing an amount of compound, preferably in the form of a solution or emulsion, from a multi-use dispenser, and/or by spraying the underside of the tongue with a preselected volume of a liquid composition, preferably a solution or emulsion, from a multi-use dispenser comprising the compound.

In one embodiment, a therapeutically effective amount of the compound is administered to the buccal vestibule inside the mouth between the cheek and the gums, preferably as a solution or emulsion, from a multi-use dispenser.

In one embodiment, the multi-use dispenser is configured for intranasal administration. In one embodiment, the multi-use dispenser is configured for administration to the oral cavity.

In one embodiment, the multi-use dispenser as described herein is for use in the treatment of neurological disease in a subject (or for use in a corresponding method of treatment), wherein the treatment comprises administering multiple doses of the self-preserving anti-microbial composition as described herein to the subject from the same dispenser.

Preferred dispensers of the invention relate to any of the above-mentioned multi-use devices. In some embodiments, the dispensers available for example from Nemera (La Verpillière, France) or Aptar Pharma (Illinois, USA) are preferred.

For example, Nemera provides Advancia® nasal spray dispensers, which are high-performing pumps with excellent dose consistency and prime retention, anti-clogging actuators, no metal contacting the formulation and hygienic anti-actuation snap-on overcaps.

As a further example, Aptar Pharma's nasal pump technology removes the need for drug manufacturers to add preservatives to nasal spray formulations. The Advanced Preservative Free (APF) systems use a tip-seal and filter technology to prevent contamination of the formulation. A spring-loaded tip seal mechanism is employed with a filter membrane in the ventilation channel.

The Nemera and Aptar technology, amongst others, offers a number of benefits, such as a metal-free fluid path thereby preventing the oxidization of the formulation, a preservative-free system and therefore an anti-microbial dispenser employing a purely mechanical barrier.

In some embodiments, the multi-use dispenser employs a membrane (preferably of silicone) that prevents bacteria from entering the reservoir or pump device. In some embodiments, the multi-use dispenser employs a metal-free fluid path, thereby preventing the oxidization of the formulation. In some embodiments, the multi-use dispenser employs a spring-loaded tip seal mechanism, thereby preventing microbes from entering the device between spray events.

Instead of using preservatives, advanced spray dispenser technology represents an alternative way of keeping a nasal spray sterile, by preventing bacteria entering and contaminating the drug formulation. Such dispensers may also be employed in the present invention, thereby further reducing microbial burden in the multi-use dispenser. In combination, the anti-microbial properties of Alpha-1062 or salts thereof with the “preservative-free”dispenser technology, as described herein, and as is available to a skilled person from e.g. Nemera or Aptar, lead to unexpectedly good shelf life (either in storage or during usage) of a liquid composition comprising Alpha-1062 or salts thereof.

Nasal sprays can be contaminated through their drug delivery orifices, with bacterial contamination coming from the external environment or the patient, or from the air. Therefore, in some embodiments, to prevent contamination via air entering the device, nasal sprays may be employed in which a filter system is present that stops the entry of bacteria into the container. Airborne bacteria are typically around 0.3 μm, therefore appropriately sized filters may be selected.

Furthermore, recent studies have demonstrated that bacterial transfer through filter membranes takes place during filtration operations, even if the pore size is significantly smaller than the bacteria size. Therefore, in some embodiments, devices are employed that utilize a silicone membrane to filter the returning air.

In one embodiment, the dispenser is configured for multiple individual spray events of 5 to 1000 μL, preferably 5 to 500 μL, more preferably 10 to 300 μL, more preferably 20 to 200 μL.

In one embodiment, the dispenser is configured for multiple individual spray events of about 5 μL, or 10, 15, 20, 25, 30, 35, 40, 45, 50, 55, 60, 65, 70, 75, 80, 85, 90, 95, 100, 120, 140, 160, 180, 200, 250, 300, 350, 400, 450 or 500 μL volume. Ranges constructed from any given of the afore-mentioned values are also contemplated.

In one embodiment, the dispenser comprises a total volume of composition of 1 to 500 mL, preferably 1 to 100 mL, more preferably 2 to 50 mL, such as for example about 5, 10 or 15 mL.

In one embodiment, the dispenser comprises about 1 mL, 2, 3, 4, 5, 6, 7, 8, 9, 10, 11, 12, 13, 14, 15, 16, 18, 20, 30, 40, 50, 60, 70, 80, 90, 100, 110, 120, 130, 140, 150, 160, 170, 180, 190, 200, 220, 240, 260, 280, 300, 350, 400, 450, or 500 mL volume. Ranges constructed from any given of the afore-mentioned values are also contemplated.

In some embodiments, the dispenser must comprise a sufficient amount of composition for administration of multiple doses. In some embodiments, the dispenser comprises a sufficient volume of composition for at least 2, or at least, or about 3, 4, 5, 6, 7, 8, 9, 10, 12, 14, 16, 18, 20, 25, 30, 35, 40, 45, 50, 60, 70, 80, 90 or about 100 individual doses, or more doses. Ranges constructed form any given of the afore-mentioned values are also contemplated.

In one embodiment, the dispenser is configured for multiple individual spray events of 20 to 200 μL, and wherein said dispenser comprises a total volume of solution of 2 to 50 mL.

In one embodiment, the multi-use dispenser is configured for and/or the Alpha-1062 or salt thereof is administered at a dose of 1 to 100 mg, one to three times daily, preferably for multiple days.

In one embodiment, the multi-use dispenser is configured for and/or the Alpha-1062 or salt thereof is administered at a dose of 2 to 40 mg, twice daily, preferably over multiple days.

In one embodiment, the multi-use dispenser is configured for and/or the Alpha-1062 or salt thereof is administered at a dosage of from 0.1 to 200 mg, 1 to 100 mg, preferably 2 to 40 mg, preferably from one to three times daily, more preferably twice daily, and even more preferably only once daily, over multiple days.

In one embodiment, the Alpha-1062 or salt thereof is administered intranasally as a 2 to 40% weight per volume (w/v) solution at an amount of 20 to 100 microliters in each of multiple nasal spray events, one to three times daily, over multiple days.

In one embodiment, the multi-use dispenser is configured for and/or the Alpha-1062 or salt thereof is administered, preferably intranasally, as an about 10% weight per volume (w/v) solution at an amount of about 50 microliters in each of multiple administration events, one to three times daily, preferably over multiple days.

In one embodiment, the multi-use dispenser is configured for and/or the Alpha-1062 or salt thereof is administered by intranasal, buccal or sublingual administration, preferably as a 2 to 40% weight per volume (w/v) solution, for example at an amount of 20 to 100 microliters, preferably multiple (intranasal or oral (sub-lingual/buccal) administration events, for example from one to three times daily, preferably over multiple days.

In one embodiment, the brain disease to be treated is Alzheimer's disease or Parkinson's Disease, and the gluconate salt of Alpha-1062 is administered intranasally, buccally or sublingually, as an about 10% weight per volume (w/v) solution at an amount of 20-100 microliters, e.g. about 50 microliters, in 1-3, preferably in a single administration event, twice daily, preferably over multiple days.

In one embodiment, the dosage regimes described herein are employed using a multi-use dispenser, as described herein. Features disclosed with respect to the dispenser are also considered disclosed with respect to the dosage regime, and vice versa.

In some embodiments, the concentrations of Alpha-1062 or salt thereof employed, preferably in the aforementioned dosage regimes, are preferably in solution or as emulsions, at about 1%, 2, 3, 4, 5, 6, 7, 8, 9, 10, 11, 12, 13, 14, 15, 16, 17, 18, 19, 20, 25, 30, 35, 40, 45, or 50 wt %, or more. Dosage regimes disclosing administration of about 10 wt %, preferably 10% solutions or emulsions, may be employed alternatively at any of the afore-mentioned concentrations, or values similar thereto. Concentration ranges constructed form any given of the afore-mentioned values are also contemplated.

In one embodiment, the compositions is in a solid form. A solid form composition may be one or more of tablets, pills, lozenges, capsules, film, or other solid composition for administration of Alpha-1062. In these embodiments, the anti-microbial properties may be beneficial either during storage, i.e. by keeping the composition free of unwanted microbes prior to administration, or may employ the anti-microbial properties of the molecule post-administration, for example when the solid form composition is administered or used to treat an infection or to kill a microorganism in another context.

A further aspect of the invention relates to a compound Alpha-1062 or salt thereof for use in the treatment of a neurological disease, wherein said subject suffers additionally from a microbial infection.

The invention therefore also relates to methods of treating a neurological disease in a subject, wherein said subject suffers additionally from a microbial infection, wherein said method comprises administering Alpha-1062 or salt thereof to a subject in need thereof.

In one embodiment, the invention relates to a compound Alpha-1062 or salt thereof for use in the treatment of a brain disease associated with cognitive impairment in a subject, wherein said subject suffers additionally from a microbial infection.

As is shown in the examples, Alpha-1062 exhibits a strong anti-microbial effect against various pathogenic microbes. As such, the compound appears ideally suited for the treatment of cognitive impairment in a subject, wherein said subject suffers additionally from a microbial infection. In some embodiments, the microbial infection is associated with said neurological disease. In some embodiments, the infection is unrelated to the neurological condition.

In one embodiment, the neurological disease to be treated is selected from Alzheimer's and/or Parkinson's disease, dementia, schizophrenia, epilepsy, stroke, poliomyelitis, neuritis, myopathy, oxygen and nutrient deficiencies in the brain after hypoxia, anoxia, asphyxia, cardiac arrest, chronic fatigue syndrome, various types of poisoning, anaesthesia, particularly neuroleptic anaesthesia, spinal cord disorders, inflammation, particularly central inflammatory disorders, postoperative delirium and/or subsyndronal postoperative delirium, neuropathic pain, abuse of alcohol and drugs, addictive alcohol and nicotine craving, and/or effects of radiotherapy.

In one embodiment, the disease to be treated is associated with cholinergic deficit. The prodrug Alpha-1062 releases galantamine in the brain upon cleavage, thereby the active agent is galantamine. Galantamine is a selective inhibitor of acetylcholinesterase (AChE), rather than butyrylcholinesterase. In addition to inhibition of AChE, galantamine interacts allosterically with nicotinic acetylcholine receptors to potentiate the action of agonists at these receptors.

In some embodiments, the invention may therefore be employed in the treatment of a medical condition associated with pathological acetylcholinesterase action, or diseases that would benefit by inhibition of AChE. In some embodiments, the invention may therefore be employed in the treatment of a medical condition associated with pathological levels or activity of nicotinic acetylcholine receptors, for example in diseases that would benefit from potentiation of nicotinic acetylcholine receptors, as caused by galantamine. A skilled person is aware of the diseases that fall into these categories or can determine them using common knowledge or routine techniques.

A further aspect of the invention therefore relates to a compound Alpha-1062 or salt thereof for use in the treatment of a microbial infection in a subject. In some embodiments, the microbial infection is a pathogenic microbial infection in a subject.

The invention therefore also relates to methods of treating a (pathogenic) microbial infection in a subject, wherein said method comprises administering Alpha-1062 or salt thereof to a subject in need thereof.

In one embodiment, the microbial infection is present in the nasal cavity, oral cavity, paranasal sinuses and/or the nasolacrimal duct.

In one embodiment, mouth infections may be treated, for example in subjects with mouth infections and cognitive disorders. Mouth infections are also known as oral infections, and are a group of infections that occur in or around the oral cavity. They include dental infection, dental abscess, and Ludwig's angina. Mouth infections typically originate from dental caries at the root of molars and premolars that spread to adjacent structures.

In one embodiment, gingivitis may be treated. The most common cause of gingivitis is the accumulation of bacterial plaque between and around the teeth. The plaque triggers an immune response, which, in turn, can eventually lead to the destruction of gingival, or gum, tissue. It may also, eventually, lead to further complications, including the loss of teeth. There are two main categories of gingival diseases, such as dental plaque-induced gingival disease: This can be caused by plaque, systemic factors, medications, or malnutrition, or non-plaque induced gingival lesions: This can be caused by a specific bacterium, virus, or fungus.

According to the preferred modes of administration, described herein, the compound will come into contact with various mucosal surfaces, which may be subject to an unwanted infection i.e. by a pathogen. The anti-microbial activity of the compound therefore provides a beneficial effect upon administration to a subject.

A further aspect of the invention relates to a method of killing a microorganism comprising administering to the microorganism an effective amount of a composition comprising Alpha-1062 or a salt thereof, preferably a liquid composition, more preferably a solution.

In one embodiment, the method is an in vitro or ex vivo method. In one embodiment, the method comprises a method of treating a subject having the microorganism to be killed.

In one embodiment, the microorganism is a bacterium, yeast or fungus. In one embodiment, the microorganism to be killed is a human pathogen.

In one embodiment, the microorganism is one or more of Escherichia coli, Pseudomonas aeruginosa, Staphylococcus aureus, Candida albicans or Aspergillus brasiliensis.

According to data provided herein, Alpha-1062 shows anti-microbial activity against both gram-positive and gram-negative bacteria, pathogenic yeasts and fungi, thereby indicating a broad-spectrum anti-microbial effect. There is no suggestion in the prior art that Alpha-1062 could exhibit such a broad and effective anti-microbial activity. This property of Alpha-1062 represents therefore a surprising and unexpected finding with practical utility.

In some embodiments, the invention relates to the use of Alpha-1062 or salt thereof as an anti-microbial. Essentially, any anti-microbial application is contemplated. In some embodiments, a pharmaceutical composition, comprising Alpha-1062 or salt thereof and/or one or more other active agents for treating neurological diseases, comprise sufficient amounts of Alpha-1062 or salt thereof to have an anti-microbial effect. In other embodiments, Alpha-1062 or salt thereof may be used to treat (disinfect) surfaces, textiles, or other objects.

A further aspect of the invention therefore relates to a method for disinfecting a pharmaceutical composition in the form of a liquid, preferably a solution, comprising bringing the liquid into contact with a compound Alpha-1062 or salt thereof.

In one embodiment, the pharmaceutical composition described herein additionally comprises one or more pharmaceutically acceptable excipients, preferably with the absence of an additional preservative.

A further aspect of the invention relates to a preparation of Alpha-1062 or salt thereof as described herein, suitable for use in preparing an anti-microbial pharmaceutical composition according to the invention. A further aspect of the invention therefore also relates to a kit, or kit-of-parts, comprising a preparation of Alpha-1062 or salt thereof useful in preparing a pharmaceutical composition. In some embodiments, such a kit may comprise one or more additional liquids or solvents, such as water, in order to prepare a solution of Alpha-1062 or salt thereof for administration to a patient.

Embodiments and features of the invention described with respect to the self-preserving composition, the multi-use dispenser, and various methods described herein, are considered to be disclosed with respect to each and every other aspect of the disclosure, such that features characterizing the methods, or dispenser, may be employed to characterize the composition, and vice-versa. The various aspects of the invention are unified by, benefit from, are based on and/or are linked by the common and surprising finding of an anti-microbial effect of Alpha-1062.

### DETAILED DESCRIPTION OF THE INVENTION

The present invention relates to a self-preserving anti-microbial pharmaceutical composition in the form of a liquid, comprising a compound Alpha-1062 or salt thereof, wherein the composition is essentially absent of additional antimicrobial preservatives. The invention further relates to a multi-use dispenser configured for transmucosal administration of a pharmaceutical composition in the form of a liquid comprising Alpha-1062 or salt thereof, in addition to various methods of using Alpha-1062 as an anti-microbial agent. In some embodiments, the composition is absent of additional antimicrobial preservatives.

The preferred molecule of the present invention is Alpha-1062, also known formerly as GLN-1062 or Memogain®. The form employed in the examples of the invention, and in one preferred embodiment of the invention, is the gluconic acid salt of Alpha-1062 (Alpha-1062 gluconate). For the sake of completeness, the compound Alpha-1062 is a galantamine pro-drug, which exhibits no or negligible activity as a cholinesterase inhibitor or nicotinic modulator prior to cleavage. Upon esterase cleavage galantamine is released.

The term “active agent” or “active pharmaceutical ingredient” (API) may however be used for Alpha-1062, as in some embodiments it is the preferred compound of the present invention. In other embodiments, the compound galantamine may also be considered an active agent or relevant drug molecule.

The chemical name (IUPAC) of Alpha-1062 is (4aS,6R,8aS)-5,6,9,10,11,12-hexahydro-3-methoxy-11-methyl-4aH-[1]benzofuro[3a,3,2-ef][2]benzazepin-6-ol benzoate.

Molecular formula of free base: C₂₄H₂₅NO₄; Molecular formula of gluconic acid: C₆H₁₂O₇; Molecular weight of free base: 391.47 g/mol; Molecular weight of Alpha-1062 gluconate: 587.61 g/mol; Conversion factor: 1 mg base=1.501 mg salt.

### Chemical Structure of Alpha-1062:

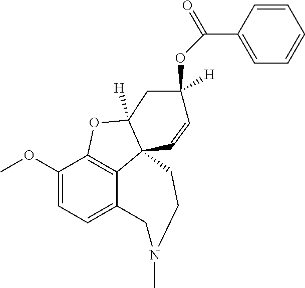

The chemical structure of the Alpha-1062 gluconate is as follows:

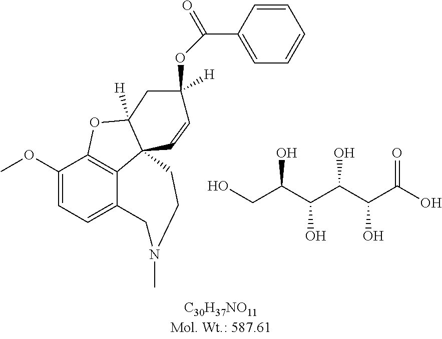

In other embodiments, galantamine is contemplated as an alternative to Alpha-1062 with respect to its anti-microbial activity. The invention therefore relates to additional aspects, wherein each embodiment disclosed herein in the context of Alpha-1062 is also contemplated for galantamine, which potentially exhibits the anti-microbial activity observed for Alpha-1062.

Galantamine has a formula of C₁₇H₂₁NO₃, and a molar mass of 287.359 g·mol⁻¹, and a structure of:

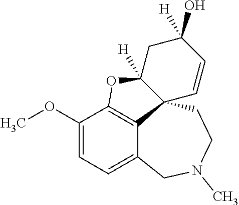

As used herein, the term “self-preserving” is a description of an anti-microbial property of a compound, namely of Alpha-1062 or salt thereof, or a composition comprising such an agent, that does not require the presence of an additional antimicrobial preserving agent.

In preferred embodiments, a self-preserving liquid composition maintains the absence of, has low or negligible numbers, or relatively slow rates of growth, of viable microbes present in the composition, or reduces the numbers of viable microbes in the composition. In some embodiments, the property “self-preserving” indicates that the rate of microbial expansion (cell growth or division) in the composition over time is lower than in the absence of the relevant compound (Alpha-1062). As used herein, a “self-preserving” liquid composition will therefore exhibit lower numbers of viable microbes in comparison to a composition without such “self-preserving” properties.

For example, a self-preserving liquid composition may be employed by a patient without significant risk due to microbial contamination for an extended period, which may extend from first use of the composition up to about 1 week, preferably 2, 3, 4, 5, 6, 7 or up to about 8 weeks. In preferred embodiments, the compositions of the invention are “self-preserving” and show no or negligible increase in the numbers of viable microbes in the composition for at least 14 days, or for at least 28 days. In some embodiments, the compositions of the invention are “self-preserving” and show no or negligible increase in the numbers of viable microbes in the composition for 1, 2, 3, 4, 5, 6, 7, 8, 9, 10, 11 or up to 12 months after first use. In some embodiments, the compositions of the invention are “self-preserving” and show no or negligible increase in the numbers of viable microbes in the composition for 1, 2, 3, 4, 5, 6, 7, 8, 9, 10, 11 or up to 12 months after manufacture and/or packaging.

In some embodiments, a “self-preserving” composition comprises no additional anti-microbial preservatives and shows no or negligible increase in the numbers of viable microbes in the composition for an extended period, preferably at least 14 days, or for at least 28 days.

As used herein, the term “anti-microbial” describes the property of a compound or composition to reduce numbers of viable microbes, or show no or a negligible increase in the numbers of viable microbes in the composition for at least 14 days, or for at least 28 days. In some embodiments, the term “anti-microbial” is defined according to the guidelines set out for preservatives in the USP 51 test. The term may therefore depend on the type of microbe and duration of test.

As used herein, the term “antimicrobial preservatives” relates to any compound that is used to reduce microbe viability in a pharmaceutical composition. Examples of such agents are, without limitation, benzalkonium chloride, benzyl alcohol, thimerosal (merthiolate), edetate disodium, monobasic sodium phosphate, providone, dibasic sodium phosphate, disodium eta, potassium phosphate monobasic, iodine, phenylcarbinol or sodium silicoaluminate. Typically, the preservative exhibits no additional pharmacological effect on e.g. a human subject, although side effects due to such preservatives have been recorded (see above).

Antimicrobial preservatives are typically substances added to nonsterile dosage forms to protect them from microbiological growth or from microorganisms that are introduced inadvertently during or subsequent to the manufacturing process. In the case of sterile articles packaged in multiple-dose dispensers, antimicrobial preservatives are usually added to inhibit the growth of microorganisms that may be introduced from repeatedly withdrawing individual doses. Typically, useful antimicrobial agents are toxic substances. For maximum protection of patients, the concentration of the preservative shown to be effective in the final packaged product should be below a level that may be toxic to human beings.

The concentration of an added antimicrobial preservative is usually kept at a minimum, or avoided completely, especially if the active ingredients of the formulation possess an intrinsic antimicrobial activity, as is the case with Alpha-1062 or salts thereof. Antimicrobial effectiveness, whether inherent in the product or whether produced because of the addition of an antimicrobial preservative, must commonly be demonstrated for multiple-dose topical and oral dosage forms and for other dosage forms such as ophthalmic, optic, nasal, irrigation, and dialysis fluids. As described herein, the agent Alpha-1062 exhibits anti-microbial preservative properties, inherent in the molecule, and therefore requires little, or no additional usage of additional preservatives in the composition of the invention.

Escherichia coli is a Gram-negative, facultative anaerobic, rod-shaped, coliform bacterium of the genus Escherichia that is commonly found in the lower intestine of warm-blooded organisms (endotherms). Most E. coli strains are harmless, but some serotypes can cause food poisoning in hosts and are occasionally responsible for food contamination. The harmless strains are part of the normal microbiota of the gut, but virulent strains can cause gastroenteritis, urinary tract infections, neonatal meningitis, hemorrhagic colitis, and Crohn's disease. Common signs and symptoms include severe abdominal cramps, diarrhea, hemorrhagic colitis, vomiting, and sometimes fever. In rarer cases, virulent strains are also responsible for bowel necrosis (tissue death) and perforation without progressing to hemolytic-uremic syndrome, peritonitis, mastitis, sepsis, and Gram-negative pneumonia. Young children are more susceptible to develop severe illness, such as hemolytic uremic syndrome; however, healthy individuals of all ages are at risk to the severe consequences that may arise as a result of being infected with E. coli. According to the present invention, Alpha-1062 and salts thereof exhibit an anti-microbial effect against E. coli, such that the invention encompasses, without limitation, reducing or limiting numbers of E. coli in liquid formulations and/or treating unwanted infections of E. coli.

Pseudomonas aeruginosa is a common encapsulated, Gram-negative, rod-shaped bacterium that can cause disease in plants and animals, including humans. A species of considerable medical importance, P. aeruginosa is typically a multidrug resistant pathogen recognized for its ubiquity, its intrinsically advanced antibiotic resistance mechanisms, and its association with serious illness, such as hospital-acquired infections, e.g. ventilator-associated pneumonia and various sepsis syndromes. The organism is considered opportunistic; serious infection often occurs during existing diseases or conditions—most notably cystic fibrosis and traumatic burns. It generally affects the immunocompromised but can also infect the immunocompetent as in hot tub folliculitis. Treatment of P. aeruginosa infections can be difficult due to its natural resistance to antibiotics. According to the present invention, Alpha-1062 and salts thereof exhibit an anti-microbial effect against P. aeruginosa, such that the invention encompasses, without limitation, reducing or limiting numbers of P. aeruginosa in liquid formulations and/or treating unwanted infections of P. aeruginosa.

Staphylococcus aureus is a Gram-positive, round-shaped bacterium that is a member of the Firmicutes, and it is a usual member of the microbiota of the body, frequently found in the upper respiratory tract and on the skin. Although S. aureus is usually unproblematic, it can also become an opportunistic pathogen, being a common cause of skin infections including abscesses, respiratory infections such as sinusitis, and food poisoning. Pathogenic strains often promote infections by producing virulence factors such as potent protein toxins. The emergence of antibiotic-resistant strains of S. aureus such as methicillin-resistant S. aureus (MRSA) is a significant problem in clinical medicine. According to the present invention, Alpha-1062 and salts thereof exhibit an anti-microbial effect against S. aureus, such that the invention encompasses, without limitation, reducing or limiting numbers of S. aureus in liquid formulations and/or treating unwanted infections of S. aureus.

Candida albicans is an opportunistic pathogenic yeast that is a common member of the human gut flora. It is detected in the gastrointestinal tract and mouth in 40-60% of healthy adults, but it can become pathogenic in immunocompromised individuals. It is one of the few species of the genus Candida that causes the human infection candidiasis, which results from an overgrowth of the fungus. C. albicans is the most common fungal species isolated from biofilms either formed on (permanent) implanted medical devices or on human tissue. A mortality rate of 40% has been reported for patients with systemic candidiasis due to C. albicans and by one estimate invasive candidiasis contracted in a hospital causes 2,800 to 11,200 deaths yearly in the US. According to the present invention, Alpha-1062 and salts thereof exhibit an anti-microbial effect against C. albicans, such that the invention encompasses, without limitation, reducing or limiting numbers of C. albicans in liquid formulations and/or treating unwanted infections of C. albicans.

Aspergillus brasiliensis, also known as Aspergillus niger, is a fungus and one of the most common species of the genus Aspergillus. It causes a disease called “black mold” on certain fruits and vegetables such as grapes, apricots, onions, and peanuts, and is a common contaminant of food. It is ubiquitous in soil and is commonly reported from indoor environments. Some strains of A. brasiliensis have been reported to produce potent mycotoxins called ochratoxins. Aspergillosis is an infection caused by A. brasiliensis. Most people breathe in Aspergillus spores every day without getting sick. However, people with weakened immune systems or lung diseases are at a higher risk of developing health problems due to Aspergillus. The types of health problems caused by Aspergillus include allergic reactions, lung infections, and infections in other organs. According to the present invention, Alpha-1062 and salts thereof exhibit an anti-microbial effect against A. brasiliensis, such that the invention encompasses, without limitation, reducing or limiting numbers of A. brasiliensis in liquid formulations and/or treating unwanted infections of A. brasiliensis.

According to data provided herein, Alpha-1062 shows anti-microbial activity against both gram-positive and gram-negative bacteria, pathogenic yeasts and fungi, thereby indicating a broad-spectrum anti-microbial effect.

In some embodiments of the invention, gram-positive bacteria can be killed or inhibited in growth by Alpha-1062 or salts thereof.

In the original bacterial phyla, the Gram-positive organisms made up the phylum Firmicutes, a name now used for the largest group. It includes many well-known genera such as Staphylococcus, Streptococcus, Enterococcus, (which are cocci) and Bacillus, Corynebacterium, Nocardia, Clostridium, Actinobacteria, and Listeria (which are rods and can be remembered by the mnemonic obconical). The gram-positive bacteria to be killed or inhibited in growth may be selected from, but is not limited to, methicillin-susceptible and methicillin-resistant staphylococci (including Staphylococcus aureus, S. epidermidis, S. haemolyticus, S. hominis, S. saprophyticus, and coagulase-negative staphy-lococci), glycopeptide intermediary-susceptible S. aureus (GISA), vancomycin-resistant Staphylococcus aureus (VRSA), penicillin-susceptible and penicillin-resistant streptococci (including Streptococcus pneumoniae, S. pyogenes, S. agalactiae, S. avium, S. bovis, S. lactis, S. sangius and Streptococci Group C, Streptococci Group G and viridans streptococci), enterococci (including vancomycin-susceptible and vancomycin-resistant strains such as Enterococcus faecalis and E. faecium), Clostridium difficile, C. clostridii-forme, C. innocuum, C. perfringens, C. ramosum, Listeria monocytogenes, Corynebacterium jelkeium, Bifidobacterium spp., Eubacterium aerofaciens, E. lentum, Lactobacillus aci-dophilus, L. casei, L. plantarum, Lactococcus spp., Leuconostoc spp., Pediococcus, Pepto-streptococcus anaeroblus, P. asaccarolyticus, P. magnus, P. micros, P. prevotii, P. productus, Propionibacterium acnes, Actinomyces spp., Moraxella spp. (including M. catarrhalis).

In some embodiments of the invention, gram-negative bacteria can be killed or inhibited in growth by Alpha-1062 or salts thereof.

Gram-negative bacteria possess a protective outer membrane consisting of lipopolysaccharides. This outer membrane protects the Gram-negative bacteria from antibiotics, dyes, and detergents that would normally damage the inner membrane or cell wall (peptidoglycan). The outer membrane provides these bacteria with resistance to lysozyme and penicillin. While alternative antimicrobial agents such as lysozyme with EDTA and the antibiotics, e.g., ampicillin, chloramphenicol, streptomycin, and nalidixic acid, have been developed to combat the protective outer membrane of some pathogenic Gram-negative microbes, the Gram-negative microbes have been evolving and becoming more immune to existing antimicrobial agents such as antibiotics.

The gram-negative bacteria to be killed or inhibited in growth may be selected from, but is not limited to, Escherichia spp. (e.g., E. Coli); Salmonella spp. (e.g., S. typhimurium); Pseudomonas spp. (e.g., P. aeruginosa); Burkholderia spp.; Neisseria spp. (e.g., N. meningitides, N. gonor-rhoeae); Haemophilus spp. (H. influenzae); Shigella spp. Bactericides spp.; Campylobacter spp.; Brucella spp.; Vibrio spp.; Yersinia spp.; Helicobacter spp.; Calymmatobacterium spp.; Legionella spp.; Leptospira spp.; Borrelia spp., Bordetella spp.; Klebsiella spp. (e.g., K 13655154.2 31 pneumoniae); Treponema spp.; Francisella spp.; Moraxella spp.; Steno-trophomonas spp.; Bdellovibrio spp.; Acinetobacter spp.; Spirochaetes; Proteus spp. (e.g., Proteus microbilis); Enterobacter; Serratia spp. (e.g., S. plymuthica, S. liquefaciens, S. rubidaea, and S. odoriferae); Gardnerella spp., and any combinations thereof. Many of these organisms are known to be pathogenic to animals and/or plants, including mammals such as humans, and can cause diseases and disorders such as enteritis, septicaemia, sepsis, meningitis, enteric fever, pneumonia, epiglottitis, cellulitis, diarrhea and sexually transmitted diseases. For example, Gram-negative cocci include three microorganisms, which cause a sexually transmitted disease (e.g., Neisseria gonorrhoeae), a meningitis (e.g., Neisseria meningitidis), and respiratory symptoms (e.g., Moraxella catarrhalis). Some of Gram-negative bacilli can cause respiratory problems (e.g., Hemophilus influenzae, Klebsiella pneumoniae, Legionella pneumophila, Pseudomonas aeruginosa), urinary prob-lems (e.g., Escherichia coli, Proteus mirabilis, Enterobacter cloacae, Serratia marcescens), and gastrointestinal problems (e.g., Helicobacter pylori, Salmonella enteritidis, Salmonella typhi). Gram-negative bacteria associated with nosocomial infections can also include, but are not limited to, Acinetobacter baumannii, which cause bacteremia, secondary meningitis, sepsis and ventilator-associated pneumonia in intensive-care units of hospital establishments.

In some embodiments of the invention, pathogenic fungi can be killed or inhibited in growth by Alpha-1062 or salts thereof.

Pathogenic fungi are fungi that cause disease in humans or other organisms. Candida species are important human pathogens that are best known for causing opportunist infections in immunocompromised hosts (e.g. transplant patients, AIDS sufferers, cancer patients). Infections are difficult to treat and can be very serious: 30-40% of systemic infections result in death. Aspergillosis is another potential fungal pathogen. Aspergillus can cause disease in three major ways: through the production of mycotoxins; through induction of allergenic responses; and through localized or systemic infections.

The pathogenic fungi to be killed or inhibited in growth may be selected from, but is not limited to Aspergillus brasiliensis, Aspergillus fumigatus, Aspergillus flavus, Aspergillus clavatus, Cryptococcus neoformans, Cryptococcus laurentii, Cryptococcus albidus, Cryptococcus gattii, Histoplasma capsulatum, Pneumocystis jirovecii, Pneumocystis carinii, Stachybotrys chartarum, Conidiobolus spp., Conidiobolus coronatus, Conidiobolus incongruous, Basidiobolus ranarum, B. dermatitidis (teleomorph, Ajellomyces dermatitidis), H. capsulatum (teleomorph, Ajellomyces capsulatus), Paracoccidioides brasiliensis, Paracoccidioides lutzii, Lacazia loboi, Coccidioides immitis, Coccidioides posadasii, and related fungi, Basidiomycete yeast, Cryptococcus gattii C. neoformans, Candida species, C. albicans, Candida glabrata, Candida parapsilosis, Candida tropicalis, Candida lusitaniae, Candida krusei, non-fumigatus species of Aspergillus, Fusarium solanum, Fusarium oxysporum, and other Fusarium spp., and their teleomorph Nectria spp., Pseudoallescheria boydii, and its Scedosporium anamorphs, Cladophialphora bantianum, Ramichloridium spp., and Dactylaria gallopava, Exophiala jeanselmei, Exophiala dermatitidis, Curvularia spp., Bipolaris, or Alternaria spp., P. jirovecii, Cryptococcus spp, Malassezia furfur, Trichosporon asahii, Rhizopus spp., Rhizopus oryzae.

As used herein, the term “multi-use dispenser”, or “multi-dose” dispenser, relates to a device or means for administration that enables the administration of multiple, individual administration actions, preferably without the necessity of opening or refilling the dispenser.

Examples of multi-use dispensers include, without limitation, those available from Nemera or Aptar, or similar devices from other manufacturers. Preferred dispensers of the invention relate to any of the above-mentioned multi-use devices mentioned herein.

According to Advancia (Nemera) technology, the intake of air into the dispenser takes place via a venting system with a silicone membrane. This technology has a continuous barrier of homogenous material which allows air to diffuse through the silicone, which acts as a permeable membrane. Consequently, the continuous barrier ensures the microbial integrity of the drug. The venting system filters the intake of air using a very fine membrane manufactured from silicone polymer. The silicone membrane is a solid, non-porous material. The membrane is homogenous and does not contain any holes. The membrane's intermolecular distance is of the order of nanometers—allowing the passage of air through the membrane but completely preventing the passage of any liquid or solid particle, including bacteria, due to the silicone membrane structure. The function of the silicone membrane can be compared to an inflated balloon. The balloon is a continuous, waterproof material yet gas slowly passes through the wall of the balloon until the pressures inside and outside reach equilibrium. Moreover, Advancia® PF offers a patented anti-clogging actuator in the upper part of the system, called closing tip. This mechanism ensures that no contamination can enter through the actuator orifice, which therefore provides protection from crystallization and clogging issues, and avoids evaporation to guarantee good prime retention.

Additional technology from Nemera is also contemplated, such as SP270+ and SP370+, which enable very good dose consistency, wide range of dose volumes: 50 μl to 200 μl with different actuators, various neck finishes: screw-on, snap-on and crimp-on, and are suitable for liquid solutions and suspensions. For unregulated markets alternative technology from Nemera is contemplated, such as SP27 and SP37. Alternatively, continuous valves for nasal and dermal delivery may be employed, such as Nemera solutions employing pressurized delivery with a neutral propellant agent (nitrogen) or liquid gases, such as CV20 for liquids, Valve 6668 for viscous products or Valve 6685 for powders.

Additional Nemera dispensing devices are disclosed, for example, in U.S. Pat. No. 9,238,532, which discloses a tip for dispensing liquid for mounting on a container. A valve includes at least two elements that are mobile in relation to one another, each mobile element including a bearing zone against the other mobile element. One of the mobile elements bears anti-microbial material on or in the immediate proximity of a part of its bearing zone forming a blocking barrier and all surfaces of the dispensing tip in contact with the inside are free of anti-microbial material. As a further example, U.S. Pat. No. 9,345,616 discloses a liquid dispenser device with air intake that reliably guarantees the sterility of the content of a reservoir. The function of taking in air and blocking air-borne micro-organisms s performed, not by an air filter, but by using the gas diffusing properties of certain materials. Thus, a member of a type other than a filter is used, namely a member made of non-porous polymer material. Such a member presents the advantage of passing non-contaminated air in a manner that is more reliable than a filter, which is porous by definition. Other technologies are disclosed in US20150043958, U.S. Pat. Nos. 8,827,124, 8,986,266, and US20140231536A1. All cited patent documents are hereby incorporated in their entirety by reference.

As further examples, Nemera provides various nasal administration technologies, such as advanced preservative free nasal pumps, classic technology platforms, CPS technology platforms, and VP3, VP6 and VP7 technologies.

As a further example, the nasal spray technology from Aptar provides excellent spray performance, is suitable for solutions, suspensions and viscous drug formulations including gels, is suitable for gamma-irradiation sterilization, and allows wide range of dose volumes from 45 μl to 1,000 μl, and offers a wide range of closures, actuators and accessories.

As a further example, the CPS technology from Aptar is a highly versatile spray pump designed for the multi-dose delivery of preserved or non-preserved drug formulations for the nasal route. CPS can be used for a range of other applications which include intra-nasal vaccination. Various benefits include wide range of dose volumes: 50 μl to 140 μl, no re-priming needed even when not used regularly, can be sterilized by irradiation. The CPS systems use anti-clogging tip seal technology to minimize crystallization with high-viscosity and high-volatile formulations, proven CPS filter technology avoids entrance of contaminated air into the container, fully validated and tested microbiological integrity, no anti-microbial additives within the pump components, and metal-free fluid pathway components.

For example, U.S. Pat. No. 9,095,864 from Aptar discloses a fluid dispenser unit and spray devices, in particular of the nasal type, and preferably applies to sprays incorporating a closure member for closing the dispenser orifice. The device mechanics disclosed therein relates to closing the dispenser orifice completely when the pump is not in use, wherein the closure member can come to co-operate directly with the dispenser orifice by being brought elastically by the return spring of the pump towards its closed position, thereby preventing any germs or bacteria penetrating inside the device between two actuations, and thereby significantly minimizing the risks of contaminating the composition to be dispensed. As another example, US20090294347 discloses a dispensing device for a liquid medium, having a medium reservoir for accommodating the medium, having a dispensing opening for dispensing the medium from the medium reservoir, and having a pressure-equalizing channel which opens out into the medium reservoir and has a microbiologically active filter arrangement inserted therein. All cited patent documents are hereby incorporated in their entirety by reference.

As used herein, the term “transmucosal administration” refers to any administration of drug, pro-drug or active agent to a mucosal membrane. Transmucosal administration means are known in the art and relate preferably to oral, nasal, vaginal, and urethral modes. The transmucosal membranes are relatively permeable, have a rich blood flow and hence allow the rapid uptake of a drug into systemic circulation to avoid first pass metabolism. The oral transmucosal delivery preferably relate to the buccal and sublingual routes. Vaginal or urethral routes can be employed using mucoadhesive suppositories, in-situ gel and foam formulations.

In some embodiments, the compositions, formulations, dispensing devices and/or pharmaceutical agents of the invention may be used in the treatment of a neurological disease.

In some embodiments, the compositions, formulations, dispensing devices and/or pharmaceutical agents of the invention may be used in the treatment of a brain disease associated with cognitive impairment.

In some embodiments, the compositions, formulations, dispensing devices and/or pharmaceutical agents of the invention may be used in the treatment of a neurodegenerative, psychiatric or neurological disease associated with a cholinergic deficit.

In some embodiments, the compositions, formulations, dispensing devices and/or pharmaceutical agents of the invention may be used in the treatment of a medical condition susceptible to treatment with a nicotinic receptor and/or a cholinergic receptor sensitizing agent.

As known in the art, three of the four presently available drugs (Donepezil, Rivastigmin, Galantamine, Memantine) are cholinergic enhancers (Donepezil, Rivastigmin, Galantamine) in that they all inhibit the family of acetylcholine-degrading enzymes denoted as cholinesterases (ChE). Inhibition of ChE increases the synaptic concentrations of acetylcholine (ACh), thereby enhancing and prolonging the action of ACh on muscarinic (mAChR) and nicotinic (nAChR) acetylcholine receptors. In addition to acting as ChE inhibitor, Galantamine (and thus Alpha-1062) also acts by allosterically stimulating (sensitizing) cholinergic receptors. Allosteric sensitization of nicotinic receptors enhances their activation by ACh or choline (Ch), thereby correcting for a disease-associated deficit in transmitter or receptor concentration (Maelicke A & Albuquerque E X (1996) Drug Discovery Today 1, 53-59; Maelicke A & Albuquerque E X (2000) Eur J Pharmacol 393, 165-170).

Examples of diseases associated with cholinergic deficit are Alzheimer's disease, Parkinson's disease, other types of dementia, schizophrenia, epilepsy, stroke, poliomyelitis, neuritis, oxygen and nutrient deficiencies in the brain after hypoxia, anoxia, asphyxia, cardiac arrest, chronic fatigue syndrome, various types of poisoning, anesthesia, spinal cord disorders, central nervous system inflammation, postoperative delirium and/or subsyndronal postoperative delirium, neuropathic pain, subsequences of the abuse of alcohol and drugs, addictive alcohol and nicotine craving, and subsequences of radiotherapy.

As used herein, the term “neurological disease” or disorder relates to any disorder of the nervous system. Structural, biochemical or electrical abnormalities in the brain, spinal cord or other nerves can result in a range of symptoms. Examples of symptoms include paralysis, muscle weakness, poor coordination, loss of sensation, seizures, confusion, pain, limitations in cognitive abilities and altered levels of consciousness. There are many recognized neurological disorders, some relatively common, but many rare. They may be assessed by neurological examination and studied and treated within the specialties of neurology and clinical neuropsychology.

Alzheimer's disease (AD), also referred to simply as Alzheimer's, is a chronic neurodegenerative disease that gradually worsens over time. It is the cause of 60-70% of cases of dementia. The most common early symptom is difficulty in remembering recent events. As the disease advances, symptoms can include problems with language, disorientation (including easily getting lost), mood swings, loss of motivation, not managing self-care, and behavioral issues.

Parkinson's disease (PD), or simply Parkinson's, is a long-term degenerative disorder of the central nervous system that mainly affects the motor system. As the disease worsens, non-motor symptoms become more common. Early in the disease, the most obvious symptoms are shaking, rigidity, slowness of movement, and difficulty with walking. Thinking and behavioral problems may also occur. Dementia becomes common in the advanced stages of the disease. The main motor symptoms are collectively called “parkinsonism”, or a “parkinsonian syndrome”.

As used herein, the term “liquid” refers to its common meaning, including compositions with nearly incompressible fluid that conforms to the shape of its container but retains a (nearly) constant volume independent of pressure.

As used herein “pharmaceutical compositions in liquid form” are liquids comprising one or more pharmaceutically active agents, suitable for administration to a subject, preferably a mammal, more preferably human subject. Liquid dosage forms are typically pharmaceutical products which involve a mixture of drug components and nondrug components (excipients). Liquid dosage forms are prepared: a) by dissolving the active drug substance in an aqueous or non-aqueous solvent (e.g. water, glycerin, ether, alcohol), or b) by suspending the drug in appropriate medium, or c) by incorporating the drug substance into an oil or water phase, such as suspensions, emulsions, syrups or elixirs. The solutions described herein are characterized by good solubility in water, as is evident for the salts of Alpha-1062.

Emulsions may also be produced and employed for transmucosal administration, as disclosed in the art. An emulsion is a mixture of two or more liquids that are normally immiscible (unmixable or unblendable). Emulsions are part of a more general class of two-phase systems of matter called colloids. Although the terms colloid and emulsion are sometimes used interchangeably, emulsion should be used when both phases, dispersed and continuous, are liquids. In an emulsion, one liquid (the dispersed phase) is dispersed in the other (the continuous phase).

Examples of emulsions include creams, ointments, liniments (balms), pastes, films, or liquids, depending mostly on their oil-to-water ratios, other additives, and their intended route of administration. Many are topical dosage forms and may be used on the surface of the skin, transdermally, transmucosally, ophthalmically, rectally or vaginally. A highly liquid emulsion may also be used orally, intranasally or may be injected in some cases.

A self-microemulsifying drug delivery system (SMEDDS) is a drug delivery system that uses a microemulsion achieved by chemical rather than mechanical means. That is, by an intrinsic property of the drug formulation, rather than by special mixing and handling. It employs the familiar ouzo effect displayed by anethole in many anise-flavored liquors. Microemulsions have significant potential for use in drug delivery. Other emulsions are generated by mechanical means, such as mixing, sonicating, vortexing or homogenizing. The first drug marketed as a SMEDD was cyclosporin, and it had significantly improved bioavailability compared with the conventional solution. SMEDDS offer numerous advantages: spontaneous formation, ease of manufacture, thermodynamic stability, and improved solubilization of bioactive materials. Improved solubility contributes to faster release rates and greater bioavailability. For many drugs taken by mouth, faster release rates improve the drug acceptance by consumers. Greater bioavailability means that less drug need be used; this may lower cost and does lower the stomach irritation and toxicity of drugs taken by mouth. For oral use, SMEDDS may be formulated as liquids, and are applicable for transmucosal administration.

The term “effective amount” or “therapeutically effective amount” used interchangeably, is defined to mean the amount or quantity of the compound (e.g. Alpha-1062), which is sufficient to elicit an appreciable biological response when administered to the patient. It will be appreciated that the precise therapeutic dose will depend on the age and condition of the patient, nature of the condition to be treated and will be at the ultimate discretion of the attendant physician. The election of dose in this case will relate to both therapeutic effect on the treatment of neurological disease, in addition to an effective amount with respect to the anti-microbial effect. Both amounts Amy be assessed and determined by a skilled person.

The pharmaceutical composition may include one or more pharmaceutically acceptable carriers, or excipients. The term “excipient” means a pharmacologically inactive component such as a diluent, disintegrant, carrier, and the like, of a pharmaceutical product. The excipients that are useful in preparing a pharmaceutical composition are generally safe, non-toxic and are acceptable for veterinary as well as human pharmaceutical use. Reference to an excipient includes both one excipient and more than one excipient. The excipients are described herein in some embodiments according to “wt %”, or “percentage by weight”.

As used herein, “administer” or “administration” refers to the delivery of the drug or agent of the present invention or a pharmaceutical composition thereof to an organism for the purpose of prevention or treatment of a brain disease associated with cognitive impairment. Suitable routes of administration may include, without limitation, oral, rectal, transmucosal or intestinal administration or intramuscular, subcutaneous, intramedullary, intrathecal, direct intraventricular, intravenous, intravitreal, intraperitoneal, intranasal, sublingual, buccal or intraocular injections. The preferred route of administration is transmucosal.

Pharmaceutical compositions of the present invention may be manufactured by processes well known in the art, e.g., by means of conventional mixing, dissolving, granulating, levigating, emulsifying, encapsulating, entrapping, dissolving or lyophilizing processes. Pharmaceutical compositions for use in accordance with the present invention may be formulated in conventional manner using one or more physiologically acceptable carriers including excipients and auxiliaries that facilitate processing the API into preparations that can be used pharmaceutically. Proper formulation is dependent upon the route of administration chosen.

For injection or transmucosal administration, the API or a pharmaceutical composition thereof may be formulated in aqueous solutions, preferably in physiologically compatible buffers such as Hanks' solution, Ringer's solution, or physiological saline buffer. For transmucosal administration, penetrants appropriate to the barrier to be permeated are used in the formulation. Such penetrants are generally known in the art.

For oral administration, the API the present invention or a pharmaceutical composition thereof can be formulated by combining the API with pharmaceutically acceptable carriers well known in the art. Such carriers enable substances, such as in crystal form, of the present invention to be formulated as tablets, pills, lozenges, capsules, liquids, gels, syrups, slurries, suspensions, solutions and the like, for oral ingestion by a patient. Pharmaceutical preparations for oral use can be made using a solid excipient, optionally grinding the resulting mixture, and processing the mixture of granules, after adding other suitable auxiliaries if desired, to obtain tablets or dragee cores. Useful excipients are, in particular, fillers such as sugars, including lactose, sucrose, mannitol, or sorbitol, cellulose preparations such as, for example, maize starch, wheat starch, rice starch and potato starch and other materials such as gelatin, gum tragacanth, methyl cellulose, hydroxypropylmethyl-cellulose, sodium carboxymethylcellulose, and/or polyvinyl-pyrrolidone (PVP). If desired, disintegrating agents may be added, such as cross-linked polyvinyl pyrrolidone, agar, or alginic acid. A salt such as sodium alginate may also be used.

A composition of the present invention may also be formulated for parenteral administration, e.g., by bolus injection or continuous infusion. Formulations for injection may be presented in unit dosage form, e.g., in ampoules or in multi-dose containers, with an added preservative. The compositions may take such forms as suspensions, solutions, or emulsions in oily or aqueous vehicles, and may contain formulating materials such as suspending, stabilizing, and/or dispersing agents.

Pharmaceutical compositions for parenteral administration include aqueous solutions of a water-soluble form of the API. Additionally, suspensions of drug or pro-drug of the present invention or pharmaceutical compositions thereof may be prepared in a lipophilic vehicle. Suitable lipophilic vehicles include fatty oils such as sesame oil, synthetic fatty acid esters such as ethyl oleate and triglycerides, or materials such as liposomes. Aqueous injection suspensions may contain substances that increase the viscosity of the suspension, such as sodium carboxymethyl cellulose, sorbitol, or dextran. Optionally, the suspension may also contain suitable stabilizers and/or agents that increase the solubility of the crystals of the present invention or a pharmaceutical composition thereof to allow for the preparation of highly concentrated solutions.

In some embodiments, the composition comprising active ingredient may be in powder form. For example, the powder may be prepared for constitution with a suitable vehicle, e.g., sterile, pyrogen-free water, before use.

In some embodiments, the powder formulation is for administration to a subject. For example, powder formulations, such as micronized powder formulations, may be administered intranasally, or via other methods, such as via transmucosal administration, by spraying or otherwise applying the powder formulation to a subject, preferably to a mucosal surface of a subject.

The antimicrobial properties of Alpha-1062 as described herein are beneficial for not only liquid formulations, such as solutions, suspensions and emulsions, but also to solid or powder formulations. Powder formulations suitable for transmucosal administration also benefit from the antimicrobial properties of Alpha-1062 and the absence of additional antimicrobial preservatives, made possible by the finding that Alpha-1062 is itself antimicrobial. Similarly, solid compositions may also benefit from the antimicrobial properties of Alpha-1062 and the absence of additional antimicrobial preservatives, leading potentially to reduced microbial contamination and longer storage times for any given Alpha-1062 composition.

A preferred mode of administration according to the present invention is transmucosal administration, i.e. through, or across, a mucous membrane. The transmucosal routes of administration of the present invention are preferably intranasal, buccal and/or sublingual. Nasal or intranasal administration relates to any form of application to the nasal cavity. The nasal cavity is covered by a thin mucosa which is well vascularized. Therefore, a drug molecule can be transferred quickly across the single epithelial cell layer without first-pass hepatic and intestinal metabolism.

Intranasal administration is therefore used as an alternative to oral administration of for example tablets and capsules, which lead to extensive degradation in the gut and/or liver. Buccal administration relates to any form of application that leads to absorption across the buccal mucosa, preferably pertaining to adsorption at the inside of the cheek, the surface of a tooth, or the gum beside the cheek. Sublingual administration refers to administration under the tongue, whereby the chemical comes in contact with the mucous membrane beneath the tongue and diffuses through it.

The compositions may, if desired, be presented in a pack or dispenser device, such as an FDA approved kit, which may contain dosage forms containing the active ingredient. The pack or dispenser device may be accompanied by instructions for administration. The pack or dispenser may also be accompanied by a notice associated with the container in a form prescribed by a governmental agency regulating the manufacture, use or sale of pharmaceuticals, which notice is reflective of approval by the agency of the form of the compositions or of human or veterinary administration. Such notice, for example, may be of the labeling approved by the U.S. Food and Drug Administration for prescription drugs or of an approved product insert.

Compositions including a compound of the present invention formulated in a compatible pharmaceutical carrier may also be prepared, placed in an appropriate container, and labeled for treatment of an indicated condition.

Suitable conditions are brain diseases associated with cognitive impairment, preferably those disclosed herein, in particular AD and PD.

### Examples

The invention is demonstrated by way of the examples disclosed herein. The examples provide technical support for a detailed description of potentially preferred, non-limiting embodiments of the invention.

The present invention is based on the surprising finding that Alpha-1062 exhibits an anti-microbial action in standard assays.

### USP 51 Assay:

In order to assess the anti-microbial properties of Alpha-1062, the USP (US Pharmacopeia) Chapter 51 Preservative Challenge Test (USP 51) was employed. The USP 51 is a common method used to gauge preservative effectiveness. Much like a Preservative Challenge Screen, it is used to evaluate the effect of preservatives in cosmetics, personal care products, and drug products. Preservatives are typically antimicrobial ingredients that are added to aqueous product formulations to help maintain the safety of the product by inhibiting the growth and reducing the number of microbial contaminants.

In the context of the present invention, Alpha-1062 (in the form of the gluconate salt) was assessed in place of (as) a preservative. No additional preservative was added to the Alpha-1062 gluconate preparation, rather an anti-microbial effect of the agent itself (Alpha-1062) was assessed. Alpha-1062 is therefore the anti-microbial agent in the assay and experiments described below.

The USP 51 challenge test utilizes 5 microorganisms (3 bacteria and 2 fungi) for challenge testing. Each of the microorganisms are known strains of pathogenic microorganisms and they represent a wide range of microbial physiologies.

For the purpose of testing under USP 51, compendial articles have been divided into four categories (see Table 1). The criteria of antimicrobial effectiveness for these products are a function of the route of administration.

TABLE 1
Compendial Product Categories

| Category | Product Description |
|---|---|
| 1 | Injections, other parenterals including emulsions, otic products, sterile nasal |
| | products, and ophthalmic products made with aqueous bases or vehicles. |
| 2 | Topically used products made with aqueous bases or vehicles, nonsterile |
| | nasal products, and emulsions, including those applied to mucous |
| | membranes. |
| 3 | Oral products other than antacids, made with aqueous bases or vehicles. |
| 4 | Antacids made with an aqueous base. |

For the present analysis, product category 2 was applied (as described below), which relates to a preferred embodiment of the invention, namely a topical nasal product to be applied transmucosally (i.e. to the mucous membrane in the nasal cavity). However, the results obtained from this analysis hold relevance for categories 1, 3 and 4, which relate to alternative embodiments of the present invention.

### Test Organisms

Cultures of the following microorganisms were applied, as per standard guidelines for USP 51:
Candida albicans (ATCC No. 10231),
Aspergillus brasiliensis (ATCC No. 16404),
Escherichia coli (ATCC No. 8739),
Pseudomonas aeruginosa (ATCC No. 9027), and
Staphylococcus aureus (ATCC No. 6538).

The viable microorganisms used in the test were not more than five passages removed from the original ATCC culture. For purposes of the test, one passage was defined as the transfer of organisms from an established culture to fresh medium. All transfers were counted. In the case of organisms maintained by seed-lot techniques, each cycle of freezing, thawing, and revival in fresh medium was taken as one transfer.

Cultures received from the ATCC were resuscitated according to standard directions. When grown in broth, the cells were pelleted by centrifugation, resuspended in 1/20th the volume of fresh maintenance broth, and added to an equal volume of 20% (v/v in water) sterile glycerol. Cells grown on agar were scraped from the surface into a 10% glycerol broth. Small aliquots of the suspension were dispensed into sterile vials, which were stored in liquid nitrogen or in a mechanical freezer. When a fresh seed-stock vial was required, it was removed and used to inoculate a series of working cultures. These working cultures were then be used as necessary (for example each day in the case of bacteria and yeast) to start the inoculum culture. All media used in the test were controlled for growth promotion.

Prior to the test, the surface of a suitable volume of solid agar medium was inoculated from a recently revived stock culture of each of the specified microorganisms. The culture conditions for the inoculum culture are described in Table 2, below, in which the suitable media are Soybean-Casein Digest or Sabouraud Dextrose Agar Medium.

To harvest the bacterial and C. albicans cultures, sterile saline TS was used to wash the surface growth and collect it in a suitable vessel. Sufficient sterile saline TS was added to obtain a microbial count of about 1×10⁸ colony-forming units (CFU) per mL. To harvest the cells of A. brasiliensis, sterile saline TS containing 0.05% of polysorbate 80 was used, and sufficient sterile saline TS was added to obtain a count of about 1×10⁸ CFU per mL.

Alternatively, the stock culture organisms were grown in a suitable liquid medium (i.e., Soybean-Casein Digest Broth or Sabouraud Dextrose Broth) and the cells harvested by centrifugation, then washed and resuspended in sterile saline TS to obtain a microbial count of about 1×10⁸ CFU per mL.

in each suspension the number of CFU per mL was determined using the conditions of media and microbial recovery incubation times listed in Table 2, below, to confirm the initial CFU per mL estimate. This value served to calibrate the size of inoculum used in the test.

### Procedure

The test was conducted in five sterile, capped bacteriological containers of suitable size into which a sufficient volume of product has been transferred. Each container was inoculated with one of the prepared and standardized inoculums and was mixed accordingly. The volume of the suspension inoculum used was between 0.5% and 1.0% of the volume of the product. The concentration of test microorganisms that was added to the product was such that the final concentration of the test preparation after inoculation was between 1×10⁵ and 1×10⁶ CFU per mL of the product, according to standard guidelines for Category 2 products.

The initial concentration of viable microorganisms in each test preparation was estimated based on the concentration of microorganisms in each of the standardized inoculums, as determined by the plate-count method.

The inoculated containers were incubated at 22.5±2.5° C. and each container was sampled at the appropriate intervals, as specified in Table 3. Any changes observed in the appearance of the culture was recorded at these intervals. By using the plate-count procedure, the number of CFU present in each test preparation was determined for the applicable intervals.

An inactivator (neutralizer) of the specific antimicrobial was incorporated in the plate count or in the appropriate dilution prepared for plating. These conditions were determined in the validation study for that sample based upon the conditions of media and microbial recovery incubation times listed in Table 2. Using the calculated concentrations of CFU per mL present at the start of the test, the change in log 10 values of the concentration of CFU per mL for each microorganism was calculated at the applicable test intervals, and the changes were expressed in terms of log reductions.

TABLE 2
Culture Conditions for Inoculum Preparation

| | | | | Microbial |
|---|---|---|---|---|
| | | | Inoculum | Recovery |
| | | Incubation | Incubation | Incubation |
| Organism | Suitable Medium | Temperature | Time | Time |
| Escherichia coli | Soybean-Casein Digest Broth; | 32.5 ± 2.5 | 18 to 24 | 3 to 5 days |
| (ATCC No. 8739) | Soybean-Casein Digest Agar | | hours | |
| Pseudomonas | Soybean-Casein Digest Broth; | 32.5 ± 2.5 | 18 to 24 | 3 to 5 days |
| aeruginosa | Soybean-Casein Digest Agar | | hours | |
| (ATCC No. 9027) | | | | |
| Staphylococcus | Soybean-Casein Digest Broth; | 32.5 ± 2.5 | 18 to 24 | 3 to 5 days |
| aureus | Soybean-Casein Digest Agar | | hours | |
| (ATCC No. 6538) | | | | |
| Candida albicans | Sabouraud Dextrose Agar; | 22.5 ± 2.5 | 44 to 52 | 3 to 5 days |
| (ATCC No. 10231) | Sabouraud Dextrose Broth | | hours | |
| Aspergillus niger | Sabouraud Dextrose Agar; | 22.5 ± 2.5 | 6 to 10 | 3 to 7 days |
| (ATCC No. 16404) | Sabouraud Dextrose Broth | | days | |

### Criteria for Antimicrobial Effectiveness

The requirements for antimicrobial effectiveness are met if the criteria specified under Table 3 are met. No increase is defined as not more than 0.5 log 10 unit higher than the previous value measured.

TABLE 3
Criteria for Antimicrobial Effectiveness
For Category 1 Products

| | |
|---|---|
| Bacteria: | Not less than 1.0 log reduction from the initial calculated count |
| | at 7 days, not less than 3.0 log reduction from the initial count |
| | at 14 days, and no increase from the 14 days' count at 28 days. |
| Yeast and Molds: | No increase from the initial calculated count at 7, 14, and 28 |
| | days. |

For Category 2 Products

| | |
|---|---|
| Bacteria: | Not less than 2.0 log reduction from the initial count at 14 days, |
| | and no increase from the 14 days' count at 28 days. |
| Yeast and Molds: | No increase from the initial calculated count at 14 and 28 days. |

For Category 3 Products

| | |
|---|---|
| Bacteria: | Not less than 1.0 log reduction from the initial count at 14 days, |
| | and no increase from the 14 days' count at 28 days. |
| Yeast and Molds: | No increase from the initial calculated count at 14 and 28 days. |

For Category 4 Products

| | |
|---|---|
| Bacteria, Yeast, | No increase from the initial calculated count at 14 and 28 days. |
| and Molds: | |

### Antimicrobial Effectiveness Report

The anti-microbial gent employed in the assay was a gluconate salt of Alpha-1062 at a concentration 82 mg/mL as a solution in water.

The data for the analysis is provided in Table 4 below:

TABLE 4
Antimicrobial Effectiveness Report for Alpha-1062 Gluconate

| | | | Day 14 | | Day 28 | |
|---|---|---|---|---|---|---|
| | | | | Log | | Log |
| | Initial | | | Re- | | Re- |
| | CFU/ | | CFU/ | duc- | CFU/ | duc- |
| Organism | mL | Log₁₀ | mL | tion | mL | tion |
| E. coli | 9.1 × 10⁵ | 6.0 | <10 | 5.0 | <10 | 5.0 |
| ATCC 8739 | | | | | | |
| P. aeruginosa | 2.9 × 10⁵ | 5.5 | <10 | 4.5 | <10 | 4.5 |
| ATCC 9027 | | | | | | |
| S. aureus | 4.0 × 10⁵ | 5.6 | <10 | 4.6 | <10 | 4.6 |
| ATCC 6538 | | | | | | |
| C. albicans | 6.7 × 10⁵ | 5.8 | <10 | 4.8 | <10 | 4.8 |
| ATCC 10231 | | | | | | |
| A. brasiliensis | 2.2 × 10⁵ | 5.3 | 1.6 × 10² | 3.1 | 1.5 × 10² | 3.1 |
| ATCC 16404 | | | | | | |

As can be seen from the data provided, the gluconate salt of Alpha-1062 exhibits a strong anti-microbial effect against all 5 organisms tested in the USP 51.

The USP Criteria for Category 2 were employed in order to determine the presence of an anti-microbial effect. For bacteria, an antimicrobial effect is evident when at least a 2.0 log reduction from the initial count is evident at 14 days and no increase is evident from the 14-day count at 28 days. For yeast and molds, an antimicrobial effect is evident when no increase from the initial calculated count is determined at 14 and 28 days.

With respect to the data above, the effect against bacteria and fungi therefore exceeded the requirements to demonstrate an anti-microbial effect for a Category 2 product.

1. A self-preserving anti-microbial pharmaceutical composition comprising a compound Alpha-1062 or salt thereof, wherein the composition is essentially absent of additional antimicrobial preservatives.

2. The self-preserving pharmaceutical composition according to claim 1, wherein the composition is in liquid form.

3. The self-preserving pharmaceutical composition according to claim 2, wherein the composition is a solution, emulsion or a suspension.

4. The self-preserving pharmaceutical composition according to claim 1, wherein the compound Alpha-1062 or salt thereof is present at a concentration of 1 to 200 mg/mL.

5. The self-preserving pharmaceutical composition according to claim 4, wherein the compound Alpha-1062 or salt thereof is present at a concentration of 5 to 100 mg/mL.

6. The self-preserving pharmaceutical composition according to claim 5, wherein the compound Alpha-1062 is present as a gluconate salt at a concentration of 50 to 100 mg/mL.

7. The self-preserving pharmaceutical composition according to claim 6, wherein the Alpha-1062 gluconate salt is present at a concentration of 70 to 90 mg/mL.

8. The self-preserving pharmaceutical composition according to claim 1, wherein the compound Alpha-1062 is present at a concentration sufficient to reduce 1×10⁴ to 1×10⁶ colony-forming units per mL (CFU/mL) of Escherichia coli, Pseudomonas aeruginosa, Staphylococcus aureus, Candida albicans and/or Aspergillus brasiliensis to <100 CFU/mL within 14 days treatment, according to the United States Pharmacopeia Chapter 51 preservative test (USP 51).

9. The self-preserving pharmaceutical composition according to claim 8, wherein the compound Alpha-1062 is present at a concentration sufficient to reduce 1×10⁴ to 1×10⁶ colony-forming units per mL (CFU/mL) of Escherichia coli, Pseudomonas aeruginosa, Staphylococcus aureus, Candida albicans and/or Aspergillus brasiliensis to <10 CFU/mL within 14 days treatment, according to the United States Pharmacopeia Chapter 51 preservative test (USP 51).

10. A multi-use dispenser configured for transmucosal administration of a pharmaceutical composition in the form of a liquid, wherein the liquid comprises the self-preserving anti-microbial composition according to claim 1.

11. The multi-use dispenser according to claim 10, wherein the dispenser is configured for intranasal administration.

12. The multi-use dispenser according to claim 10, wherein the dispenser is configured for administration in the oral cavity.

13. The multi-use dispenser according to claim 10, wherein the dispenser is configured for multiple individual administration events of 10 to 300 μL and wherein said dispenser comprises a total volume of liquid composition of 1 to 100 mL.

14. The multi-use dispenser according to claim 13, wherein the dispenser is configured for multiple individual administration events of 20 to 200 μL and wherein said dispenser comprises a total volume of liquid composition of 2 to 50 mL.

15. A method for the treatment of a neurological disease in a subject, wherein said treatment comprises administering multiple doses of the self-preserving anti-microbial composition according to claim 1 to the subject from the same dispenser.

16. A method for the treatment of a neurological disease in a subject, comprising administering a compound Alpha-1062 or salt thereof to the subject, wherein said subject suffers additionally from a microbial infection.

17. A method for the treatment of a microbial infection in a subject, comprising administering a compound Alpha-1062 or salt thereof to the subject.

18. The method according to claim 16, wherein the microbial infection is present in the nasal cavity, oral cavity, paranasal sinuses and/or the nasolacrimal duct of the subject.

19. A method of killing a microorganism comprising contacting the microorganism with an effective amount of a composition comprising a compound Alpha-1062 or a salt thereof.

20. The method according to claim 19, wherein the microorganism is a bacterium, yeast or fungus.

21. The method according to claim 19, wherein the microorganism is a human pathogen.

22. The method according to claim 19, wherein the microorganism is Escherichia coli, Pseudomonas aeruginosa, Staphylococcus aureus, Candida albicans and/or Aspergillus brasiliensis.

23. The method according to claim 17, wherein the microbial infection is present in the nasal cavity, oral cavity, paranasal sinuses and/or the nasolacrimal duct of the subject.

24. A self-preserving anti-microbial pharmaceutical composition according to claim 1, wherein the composition is absent of additional antimicrobial preservatives and is in the form of a liquid, wherein the composition is present in a multi-use dispenser configured for intranasal administration, wherein the dispenser is configured for multiple individual administration events of 10 to 300 μL, and wherein said dispenser comprises a total volume of liquid composition of 1 to 100 mL.
